(19) United States
(12) Patent Application Publication
(10) Pub. No.: US 2015/0238260 A1
(43) Pub. Date: Aug. 27, 2015

(54) SURGICAL INSTRUMENTS INCLUDING NERVE STIMULATOR APPARATUS FOR USE IN THE DETECTION OF NERVES IN TISSUE AND METHODS OF DIRECTING ENERGY TO TISSUE USING SAME

(71) Applicant: COVIDIEN LP, MANSFIELD, MA (US)

(72) Inventor: WILLIAM H. NAU, JR., LONGMONT, CO (US)

(21) Appl. No.: 14/546,695

(22) Filed: Nov. 18, 2014

Related U.S. Application Data
(60) Provisional application No. 61/944,614, filed on Feb. 26, 2014.

(51) Int. Cl. A61B 18/22; A61B 17/28

(52) U.S. Cl. CPC A61B 18/22; A61B 17/282

(57) ABSTRACT

A forceps includes a housing a shaft including a distal end and a proximal end operatively coupled to the housing, and an end-effector assembly coupled to the distal end of the shaft and including first and second jaw assemblies. Each of the first and second jaw assemblies includes a sealing plate. One or both of the first and second jaw assemblies is movable from a spaced relation relative to the other jaw assembly to at least one subsequent position wherein the sealing plates cooperate to grasp tissue therebetween. The forceps also includes a nerve stimulator apparatus associated with one or both of the first and second jaw assemblies. The nerve stimulator apparatus is configured to emit light to stimulate tissue for the evaluation of one or more characteristics of nerves.

### CROSS REFERENCE TO RELATED APPLICATION

The present application claims the benefit of and priority to U.S. Provisional Application Ser. No. 61/944,614, filed on Feb. 26, 2014, the entire contents of which are incorporated herein by reference.

### BACKGROUND

1. Technical Field

The present disclosure relates to surgical systems and devices for performing medical procedures. The present disclosure also relates to optical detection devices for use in connection with surgical devices. More particularly, the present disclosure relates to surgical systems and surgical instruments, such as, for example, a vessel-sealing device, including a nerve stimulator apparatus for use in the detection of nerves in tissue, and which may be useful for testing and/or monitoring the viability and functionality of nerves. The present disclosure also relates to methods of treating tissue using the same.

2. Discussion of Related Art

Electrosurgical instruments have become widely used by surgeons. Electrosurgery involves the application of thermal and/or electrical energy to cut, dissect, ablate, coagulate, cauterize, seal or otherwise treat biological tissue during a surgical procedure. Electrosurgery is typically performed using an electrosurgical generator operable to output energy and a handpiece including a surgical instrument (e.g., end effector) adapted to transmit energy to a tissue site during electrosurgical procedures. Electrosurgery can be performed using either a monopolar or a bipolar instrument.

The basic purpose of both monopolar and bipolar electrosurgery is to produce heat to achieve the desired tissue/clinical effect. In monopolar electrosurgery, devices use an instrument with a single, active electrode to deliver energy from an electrosurgical generator to tissue, and a patient return electrode or pad that is attached externally to the patient (e.g., a plate positioned on the patient's thigh or back) as the means to complete the electrical circuit between the electrosurgical generator and the patient. When the electrosurgical energy is applied, the energy travels from the active electrode, to the surgical site, through the patient and to the return electrode. In bipolar electrosurgery, both the active electrode and return electrode functions are performed at the site of surgery. Bipolar electrosurgical devices include two electrodes that are located in proximity to one another for the application of current between their surfaces. Bipolar electrosurgical current travels from one electrode, through the intervening tissue to the other electrode to complete the electrical circuit. Bipolar instruments generally include end-effectors, such as grippers, cutters, forceps, dissectors and the like.

Forceps utilize mechanical action to constrict, grasp, dissect and/or clamp tissue. By utilizing an electrosurgical forceps, a surgeon can utilize both mechanical clamping action and electrosurgical energy to effect hemostasis by heating the tissue and blood vessels to cauterize, coagulate/desiccate, seal and/or divide tissue. Bipolar electrosurgical forceps utilize two generally opposing electrodes that are operably associated with the inner opposing surfaces of end effectors and that are both electrically coupled to an electrosurgical generator. In bipolar forceps, the end-effector assembly generally includes opposing jaw assemblies pivotably mounted with respect to one another. In bipolar configuration, only the tissue grasped between the jaw assemblies is included in the electrical circuit. Because the return function is performed by one jaw assembly of the forceps, no patient return electrode is needed.

By utilizing an electrosurgical forceps, a surgeon can cauterize, coagulate/desiccate and/or seal tissue and/or simply reduce or slow bleeding by controlling the intensity, frequency and duration of the electrosurgical energy applied through the jaw assemblies to the tissue. During the sealing process, mechanical factors such as the pressure applied between opposing jaw assemblies and the gap distance between the electrically-conductive tissue-contacting surfaces (electrodes) of the jaw assemblies play a role in determining the resulting thickness of the sealed tissue and effectiveness of the seal.

The term “thermal spread” refers generally to the heat transfer (e.g., heat conduction, heat convection, or electrical current dissipation) dissipating along the periphery of the electrically-conductive or electrically-active surfaces of an electrosurgical instrument to adjacent tissue. The reduction and control of thermal spread to surrounding tissues during an electrosurgical procedure reduces the likelihood of unintentional and/or undesirable collateral damage to surrounding tissue structures, e.g., nerve tissue, which may be adjacent to an intended treatment site.

### SUMMARY

Patients may suffer from complications as a result of nerve damage during surgery. Symptoms associated with nerve damage are dependent upon the location, type of nerve, and the severity of the damage, and may result in loss of function, weakness, muscle atrophy, fasciculation, paralysis, cardiac irregularities, allodynia, and chronic neuropathy. The cause of nerve damage during surgical procedures varies but is often the result of inadvertent surgical damage due to poor visibility of the nerve as compared to surrounding tissues. In some cases, nerve damage may be unavoidable due to close proximity of the nerve to target structures.

According to an aspect of the present disclosure, a forceps provided. The forceps includes a housing a shaft including a distal end and a proximal end operatively coupled to the housing, and an end-effector assembly coupled to the distal end of the shaft and including first and second jaw assemblies. Each of the first and second jaw assemblies includes a sealing plate. One or both of the first and second jaw assemblies is movable from a spaced relation relative to the other jaw assembly to at least one subsequent position wherein the sealing plates cooperate to grasp tissue therebetween. The forceps also includes a nerve stimulator apparatus associated with one or both of the first and second jaw assemblies. The nerve stimulator apparatus is configured to emit light to stimulate tissue for the detection and/or evaluation of one or more characteristics and/or properties of nerves.

According to another aspect of the present disclosure, a method of treating tissue is provided. The method includes the initial step of positioning an end-effector assembly including first and second jaw assemblies at a first position within tissue. Each of the first and second jaw assemblies includes a sealing plate. One or both of the first and second jaw assemblies is movable from a spaced relation relative to the other jaw assembly to at least one subsequent position wherein the sealing plates cooperate to grasp tissue therebetween. The method also includes the steps of activating a nerve stimulator apparatus associated with one or both of the first and second jaw assemblies to emit light to stimulate target tissue, and determining nerve proximity relative to the first position of the end-effector assembly by measuring one or more characteristics of nerves within the target tissue based on a response to light entering the target tissue.

In any one of the preceding aspects, one or more characteristics of nerves may include location, viability and functionality of the nerves. In any one of the preceding aspects, evaluation of one or more characteristics and/or properties of nerves may include: detecting and/or monitoring changes in blood pressure, heart rate, and/or breathing rate; detecting muscle contraction and/or twitches; and/or detecting and/or monitoring the release of one or more hormones (and/or other biochemicals).

According to another aspect of the present disclosure, a method of treating tissue is provided. The method includes the initial step of positioning an end-effector assembly including first and second jaw assemblies at a first position within tissue. Each of the first and second jaw assemblies includes an outer housing and a sealing plate. One or both of the first and second jaw assemblies is movable from a spaced relation relative to the other jaw assembly to at least one subsequent position wherein the sealing plates cooperate to grasp tissue therebetween. The method also includes the steps of activating a nerve stimulator apparatus associated with an outer housing of one or both of the first and second jaw assemblies to emit light to stimulate target tissue, measuring one or more properties of backscattered light from internal microstructure in the target tissue to make a determination of nerve proximity relative to the first position of the end-effector assembly, and determining whether to move the end-effector assembly from the first position to a second position based at least in part on the determination of nerve proximity relative to the first position of the end-effector assembly.

### BRIEF DESCRIPTION OF THE DRAWINGS

Objects and features of the presently-disclosed end-effector assemblies including a nerve stimulator apparatus for use in surgical instruments, systems including the same, and methods of treating tissue using the same of the present disclosure will become apparent to those of ordinary skill in the art when descriptions of various embodiments thereof are read with reference to the accompanying drawings, of which:

FIG. 1 is a perspective view of an endoscopic bipolar forceps including a housing, a rotatable member, a shaft, and an end-effector assembly that includes a nerve stimulator apparatus in accordance with an embodiment of the present disclosure;

FIG. 2 is a perspective view of an endoscopic bipolar forceps including a housing, a rotatable member, a shaft, and an end-effector assembly that includes a nerve stimulator apparatus in accordance with another embodiment of the present disclosure;

FIG. 3 is a perspective view of an open surgical forceps including first and second shafts and an end-effector assembly in accordance with an embodiment of the present disclosure;

FIG. 4 is an enlarged, perspective view of a distal portion of the shaft and the end-effector assembly of the endoscopic bipolar forceps shown in FIG. 1 in accordance with an embodiment of the present disclosure;

FIG. 5 is an enlarged, perspective view of an end-effector assembly that includes nerve stimulator apparatus including a selectively-translatable fiber-optical nerve stimulation member in accordance with an embodiment of the present disclosure;

FIG. 6A is a side, cross-sectional view of an end-effector assembly in accordance with an embodiment of the present disclosure;

FIG. 6B is a front, cross-sectional view of the end-effector assembly shown in FIG. 6A;

FIG. 7 is a side, schematic view of a laser fiber of the end-effector assembly shown in FIG. 6A;

FIG. 8A is an enlarged, perspective view of a distal portion of an endoscopic surgical instrument including an end-effector assembly that includes a nerve stimulator apparatus in accordance with an embodiment of the present disclosure;

FIG. 8B is an end, schematic view of the end-effector assembly shown in FIG. 8A;

FIG. 9 is a flowchart illustrating a method of treating tissue in accordance with an embodiment of the present disclosure; and

FIG. 10 is a flowchart illustrating a method of treating tissue in accordance with another embodiment of the present disclosure.

### DETAILED DESCRIPTION

Hereinafter, embodiments of end-effector assemblies including a nerve stimulator apparatus for use in surgical instruments, systems including the same, and methods of treating tissue using the same of the present disclosure are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the apparatus, or component thereof, closer to the user and the term “distal” refers to that portion of the apparatus, or component thereof, farther from the user.

This description may use the phrases “in an embodiment,” “in embodiments,” “in some embodiments,” or “in other embodiments,” which may each refer to one or more of the same or different embodiments in accordance with the present disclosure.

A laser is a device that emits light through a process of optical amplification based on the stimulated emission of electromagnetic radiation. A laser may be classified as operating in either continuous or pulsed mode, depending on whether the power output is essentially continuous over time or whether its output takes the form of pulses of light.

Nerve damage can be caused by a wide variety of reasons. Damage to nerves can be caused by physical injury, swelling, autoimmune diseases, infection, diabetes, failure of the blood vessels surrounding the nerve, or other medical conditions. Unintentional nerve damage can occur during surgical operations, e.g., cutting and/or sealing. In some cases, nerve damage can be caused by thermal spread during an electrosurgical procedure.

Various embodiments of the present disclosure provide surgical instruments suitable for sealing, cauterizing, coagulating/desiccating and/or cutting vessels and vascular tissue. Various embodiments of the present disclosure provide surgical instruments including a nerve stimulator apparatus configured to emit light to stimulate tissue for the detection and/or evaluation of one or more characteristics and/or properties of nerves. Embodiments of the presently-disclosed nerve stimulator apparatus may be suitable for use for testing and/or monitoring the viability and functionality of nerves, e.g., prior to, during, and/or after the application of energy to tissue during a surgical procedure.

Various embodiments of the present disclosure provide a forceps with an end-effector assembly including a nerve stimulator apparatus configured to emit light to stimulate tissue for the detection of nerves, or testing and/or monitoring the viability and functionality of nerves. Embodiments of the presently-disclosed nerve stimulator apparatus include one or more optical stimulator devices, which may be configured to emit light in the form of optical pulses, continuous-wave laser irradiation, and/or other forms of light. Embodiments of the presently-disclosed optical stimulator devices may include ultraviolet lasers, infrared lasers, pulsed lasers, gas lasers, solid-state lasers, diode lasers, infrared pulsed diode lasers, and/or other devices suitable for effecting optical nerve stimulation.

Embodiments of the presently-disclosed forceps may be suitable for utilization in endoscopic surgical procedures and/or suitable for utilization in open surgical applications. Embodiments of the presently-disclosed bipolar forceps may be implemented using electromagnetic radiation at radio frequencies (RF) or at other frequencies.

The various embodiments disclosed herein may also be configured to work with robotic surgical systems and what is commonly referred to as “Telesurgery”. Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

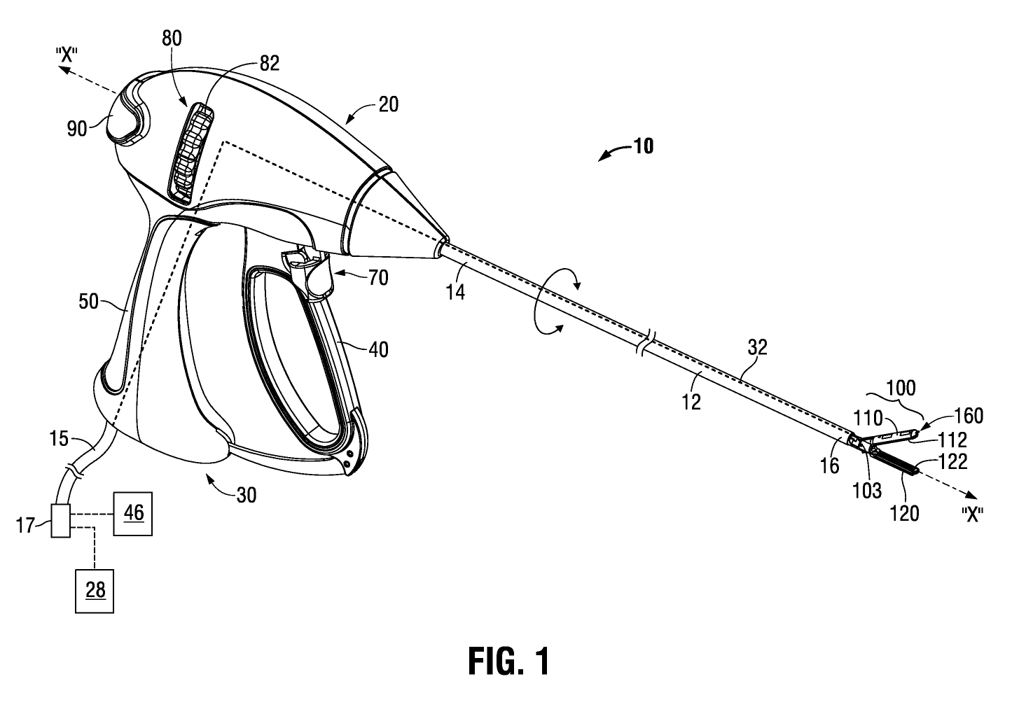

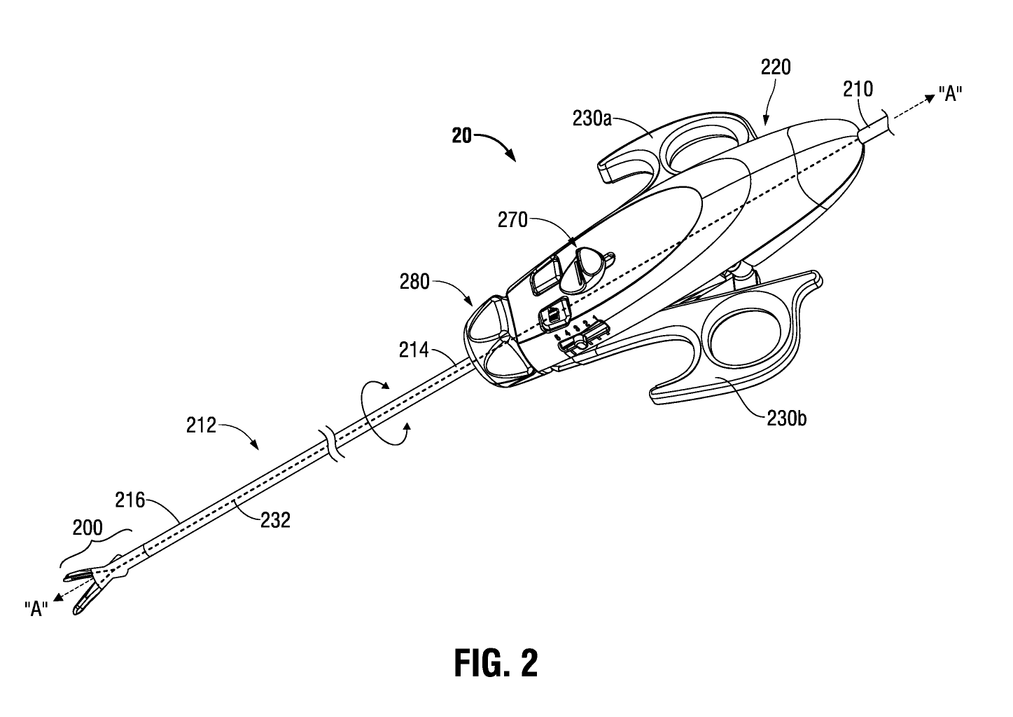

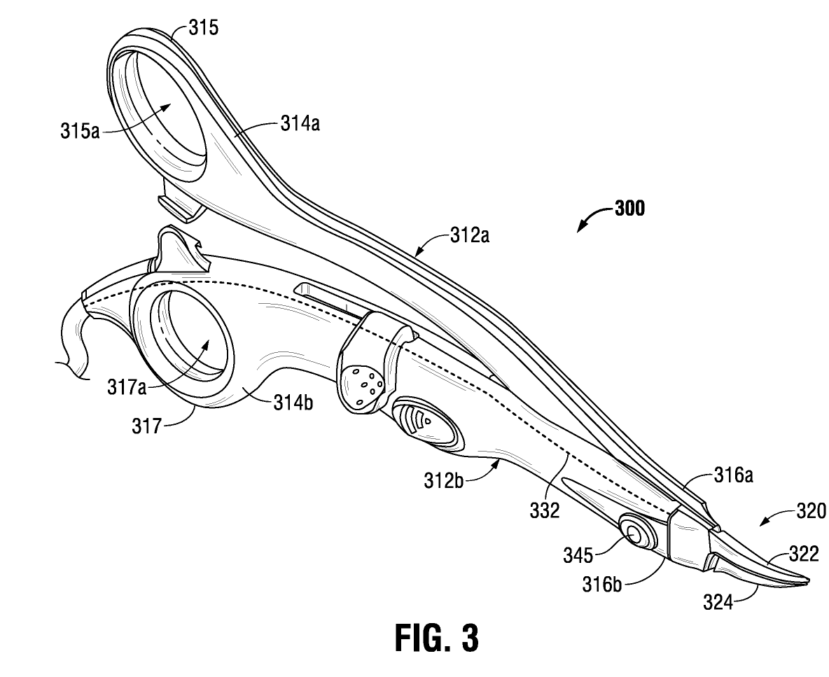

The vessel-sealing instruments illustrated in FIGS. 1-3 are three examples of a family of surgical instruments used for tissue fusion. FIGS. 1 and 2 depict two embodiments of a bipolar forceps for use in connection with endoscopic surgical procedures, and an open version of a bipolar forceps is shown in FIG. 3. Although the following description describes the use of bipolar forceps, the teachings of the present disclosure may also apply to a variety of surgical instruments, e.g., surgical staplers, wherein the determination of nerve proximity and/or testing and/or monitoring of the viability and functionality of nerves during a variety of procedures and operations may improve outcomes.

In FIG. 1, an endoscopic bipolar forceps 10 is shown for use with various surgical procedures and includes a housing 20, a handle assembly 30, a rotatable assembly 80, a trigger assembly 70, and an end-effector assembly 100, which mutually cooperate to grasp, seal and/or divide tissue, e.g., tubular vessels and vascular tissue. End-effector assembly 100 includes a nerve stimulator apparatus 160 configured to emit light to stimulate tissue for detection and/or evaluation of one or more characteristics and/or properties of nerves, e.g., prior to, during, and/or after the application of energy to tissue. In some embodiments, the evaluation of one or more characteristics and/or properties of nerves may include testing and/or monitoring the viability and functionality of nerves. In accordance with another embodiment of the present disclosure, a bipolar forceps (shown generally as 20 in FIG. 2) for use with endoscopic surgical procedures includes two movable handles 230a and 230b disposed on opposite sides of a housing 220, a rotatable assembly 280, a knife trigger assembly 270, and an end-effector assembly 200. For the purposes herein, the forceps 10 and 20 are described in terms of an endoscopic instrument; however, an open version of the forceps (e.g., bipolar forceps 300 shown in FIG. 3) may also include the same or similar operating components and features as described below.

Forceps 10 includes a shaft 12 having a distal end 16 configured to mechanically engage the end-effector assembly 22 and a proximal end 14 configured to mechanically engage the housing 20. End-effector assembly 100 may be selectively and releaseably engageable with the distal end 14 of the shaft 12, and/or the proximal end 16 of the shaft 12 may be selectively and releaseably engageable with the housing 20 and the handle assembly 30.

The proximal end 14 of the shaft 12 is received within the housing 20, and connections relating thereto are disclosed in commonly assigned U.S. Pat. No. 7,150,097 entitled “METHOD OF MANUFACTURING JAW ASSEMBLY FOR VESSEL SEALER AND DIVIDER,” commonly assigned U.S. Pat. No. 7,156,846 entitled “VESSEL SEALER AND DIVIDER FOR USE WITH SMALL TROCARS AND CANNULAS,” commonly assigned U.S. Pat. No. 7,597,693 entitled “VESSEL SEALER AND DIVIDER FOR USE WITH SMALL TROCARS AND CANNULAS” and commonly assigned U.S. Pat. No. 7,771,425 entitled “VESSEL SEALER AND DIVIDER HAVING A VARIABLE JAW CLAMPING MECHANISM.”

Forceps 10 includes a cable 15. Cable 15 may be formed from a suitable flexible, semi-rigid, or rigid cable, and may connect directly to a power generating source 28. In some embodiments, the cable 15 connects the forceps 10 to a connector 17, which further operably connects the forceps 10 to the power generating source 28, and which may further connect the instrument 10 to a laser light source 46, e.g., an infrared light source. Cable 15 may be internally divided into one or more cable leads each of which transmits energy through their respective feed paths to the end-effector assembly 100. Cable 15 may include optical fiber 32 which transmits light to the nerve stimulator apparatus 160.

Power generating source 28 may be any generator suitable for use with surgical devices, and may be configured to provide various frequencies of electromagnetic energy. Examples of generators that may be suitable for use as a source of energy are commercially available under the trademarks FORCE EZ™, FORCE FX™, and FORCE TRIAD™ offered by Covidien Surgical Solutions of Boulder, Colo. Forceps 10 may alternatively be configured as a wireless device or battery-powered.

End-effector assembly 100 generally includes a pair of opposing jaw assemblies 110 and 120 pivotably mounted with respect to one another. End-effector assembly 100 may be configured as a bilateral jaw assembly, i.e., both jaw assemblies 110 and 120 move relative to one another. Alternatively, the forceps 10 may include a unilateral assembly, i.e., the end-effector assembly 100 may include a stationary or fixed jaw assembly, e.g., 120, mounted in fixed relation to the shaft 12 and a pivoting jaw assembly, e.g., 110, mounted about a pivot pin 103 coupled to the stationary jaw assembly. Jaw assemblies 110 and 120 may be curved at various angles to facilitate manipulation of tissue and/or to provide enhanced line-of-sight for accessing targeted tissues.

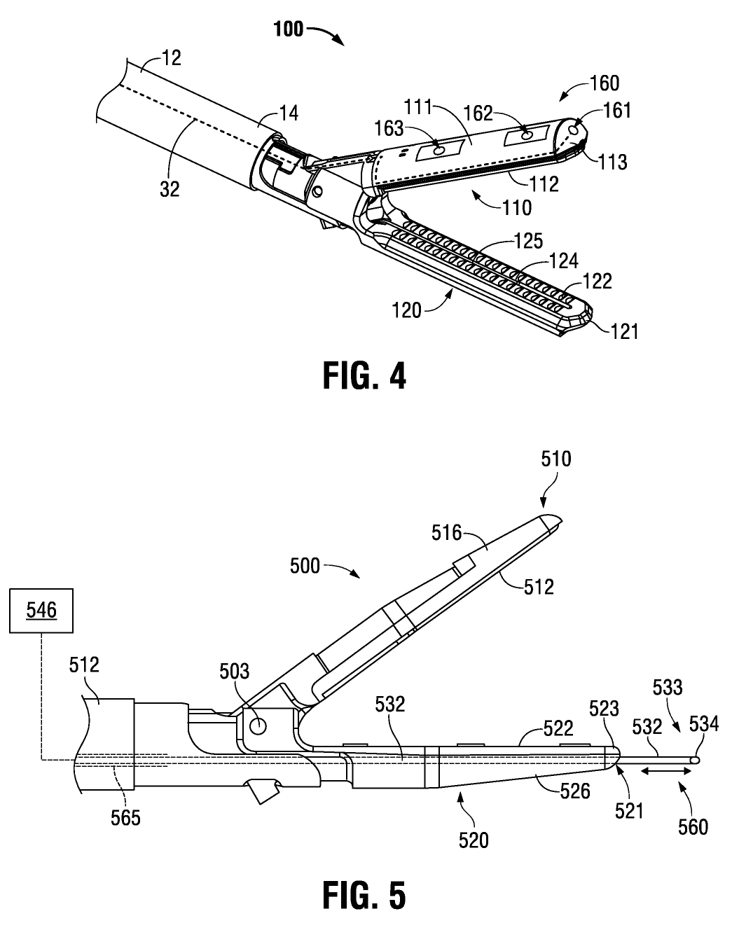

Jaw assemblies 110 and 120, as shown in FIGS. 1 and 4, include an electrically-conductive tissue-engaging surface or sealing plate 112 and 122, respectively, arranged in opposed relation relative to one another and associated with an outer housing 111 and 121, respectively (FIG. 4). In some embodiments, the outer housings 111 and 121 define a cavity therein configured to at least partially encapsulate and/or securely engage the sealing plates 112 and 122, respectively, and/or other jaw assembly components. As described in more detail later in this description, various components of the nerve stimulator apparatus 160 are associated with the outer housing 111 and/or the cavity defined therein. The outer housings 111 and 121 may be formed, at least in part, of a non-electrically-conductive or substantially non-electrically-conductive material. In some embodiments, the outer housing 111 and 121 may include ceramic or any of a variety of suitable non-electrically conductive materials such as polymeric materials, e.g., plastics, and/or other insulative materials.

One or both of the jaw assemblies 110 and 120 include a longitudinally-oriented slot or knife channel configured to permit reciprocation of a knife blade (not shown). In some embodiments, as shown in FIG. 4, the knife channel 125 may be completely disposed in one of the two jaw assemblies, e.g., jaw assembly 120, depending upon a particular purpose.

Examples of sealing plate 112, 122, outer housing 111, 121, and knife blade embodiments are disclosed in commonly assigned International Application Serial No. PCT/US01/11412 filed on Apr. 6, 2001, entitled “ELECTROSURGICAL INSTRUMENT WHICH REDUCES COLLATERAL DAMAGE TO ADJACENT TISSUE,” and commonly assigned International Application Serial No. PCT/US01/11411 filed on Apr. 6, 2001, entitled “ELECTROSURGICAL INSTRUMENT REDUCING FLASHOVER.”

As shown in FIG. 1, the end-effector assembly 22 is rotatable about a longitudinal axis “X-X” through rotation, either manually or otherwise, of the rotatable assembly 80. Rotatable assembly 80 generally includes two halves (not shown), which, when assembled about a tube of shaft 12, form a generally circular rotatable member 82. Rotatable assembly 80, or portions thereof, may be configured to house a drive assembly (not shown) and/or a knife assembly (not shown), or components thereof. A reciprocating sleeve (not shown) is slidingly disposed within the shaft 12 and remotely operable by the drive assembly (not shown). Examples of rotatable assembly embodiments, drive assembly embodiments, and knife assembly embodiments of the forceps 10 are described in the above-mentioned, commonly-assigned U.S. Pat. Nos. 7,150,097, 7,156,846, 7,597,693 and 7,771,425.

Handle assembly 30 includes a fixed handle 50 and a movable handle 40. In some embodiments, the fixed handle 50 is integrally associated with the housing 20, and the movable handle 40 is selectively movable relative to the fixed handle 50. Movable handle 40 of the handle assembly 30 is ultimately connected to the drive assembly (not shown). As can be appreciated, applying force to move the movable handle 40 toward the fixed handle 50 pulls the drive sleeve (not shown) proximally to impart movement to the jaw assemblies 110 and 120 from an open position, wherein the jaw assemblies 110 and 120 are disposed in spaced relation relative to one another, to a clamping or closed position, wherein the jaw assemblies 110 and 120 cooperate to grasp tissue therebetween. Examples of handle assembly embodiments of the forceps 10 are described in the above-mentioned, commonly-assigned U.S. Pat. Nos. 7,150,097, 7,156,846, 7,597,693 and 7,771,425.

Forceps 10 includes a switch 90 configured to permit the user to selectively activate the forceps 10 in a variety of different orientations, i.e., multi-oriented activation. As can be appreciated, this simplifies activation. When the switch 90 is depressed, energy is transferred through one or more electrical leads to the jaw assemblies 110 and 120. Although FIG. 1 depicts the switch 90 disposed at the proximal end of the housing assembly 20, switch 90 may be disposed on another part of the forceps 10 (e.g., the fixed handle 50, rotatable member 82, etc.) or another location on the housing assembly 20.

Turning now to FIG. 2, forceps 20 generally includes a shaft 212 that has a distal end 216 configured to mechanically engage the end-effector assembly 200 and a proximal end 214 that mechanically engages the housing 220. End-effector assembly 200 may include any feature or combination of features of the nerve stimulator apparatus embodiments disclosed herein. Forceps 20 generally includes optical fiber 232, which extends through the shaft 212 to the end-effector assembly 200.

Forceps 20 includes a cable 210 that connects the forceps 20 to a source of energy (e.g., power generating source 28 shown in FIG. 1). Cable 210 may include optical fiber 232 for use to transmit light to any of the nerve stimulator apparatus embodiments disclosed herein. Handles 230a and 230b disposed on opposite sides of housing 220 are movable relative to one another to actuate the end-effector assembly 200.

Rotatable assembly 280 is mechanically coupled to the housing 220 and is rotatable approximately 90 degrees in either direction about a longitudinal axis “A-A” defined through the shaft 212. Rotatable assembly 280, when rotated, rotates the shaft 212, which, in turn, rotates the end-effector assembly 200. Such a configuration allows the end-effector assembly 200 to be rotated approximately 90 degrees in either direction with respect to the housing 220. The details of the inner-working components of forceps 20 are disclosed in commonly-owned U.S. Pat. No. 7,789,878 entitled “IN-LINE VESSEL SEALER AND DIVIDER.”

In FIG. 3, an embodiment of an open forceps 300 is shown for use with various surgical procedures and generally includes a pair of opposing shafts 312a and 312b having an end-effector assembly 320 attached to the distal ends 316a and 316b thereof, respectively. End-effector assembly 320 includes a pair of opposing jaw members 322 and 324 that are pivotably connected about a pivot pin 365 and movable relative to one another to grasp tissue. Forceps 300 includes optical fiber 332, e.g., associated with at least one of the shafts (e.g., shaft 312b), suitable for transmitting light to any of the nerve stimulator apparatus embodiments disclosed herein.

Each shaft 312a and 312b includes a handle 315 and 317, respectively, disposed at the proximal end 314a and 314b thereof, respectively. Each handle 315 and 317 defines a finger and/or thumb hole 315a and 317a, respectively, therethrough for receiving the user's finger or thumb. Finger and/or thumb holes 315a and 317a facilitate movement of the shafts 312a and 312b relative to one another to pivot the jaw members 322 and 324 from an open position, wherein the jaw members 322 and 324 are disposed in spaced relation relative to one another, to a clamping or closed position, wherein the jaw members 322 and 324 cooperate to grasp tissue therebetween. End-effector assembly 320 may include any feature or combination of features of the nerve stimulator apparatus embodiments disclosed herein.

FIG. 4 shows the end-effector assembly 100 of the endoscopic bipolar forceps 10 shown in FIG. 1, including opposing jaw assemblies 110 and 120. As depicted in FIGS. 1 and 4, the end-effector assembly 100 includes the nerve stimulator apparatus 160. Nerve stimulator apparatus 160 is configured to emit light to stimulate tissue for detection of nerves and/or evaluation of one or more characteristics and/or properties of nerves, which may include, for example, testing and/or monitoring the viability and functionality of nerves, e.g., prior to, during, and/or after the application of energy to tissue. In some embodiments, testing and/or monitoring the viability and functionality of nerves may include detecting and/or monitoring changes in blood pressure (and/or heat rate), detecting muscle contraction and/or twitches, and/or detecting and/or monitoring the release of one or more hormones (and/or other biochemicals). In some embodiments, the detection of nerves may include detecting changes in one or more optical properties of the nerves (e.g., fluorescence or absorbance), and may include the use of one or more intraoperative imaging modalities to allow for intraoperative visualization of sensitive structures, e.g., nerves.

Nerve stimulator apparatus 160 may include one or more optical stimulator devices associated with any of the various components of the jaw assembly 110 and/or the jaw assembly 120. In some embodiments, as shown in FIG. 4, the nerve stimulator apparatus 160 includes three optical stimulator devices 161, 162 and 163 associated with the jaw assembly 110. Optical stimulator devices 161, 162 and 163 may include any device suitable for effecting optical nerve stimulation, and may be configured to emit light in the form of optical pulses, continuous-wave laser irradiation, and/or other forms of light. In some embodiments, the optical stimulator devices 161, 162 and 163 may include ultraviolet lasers, infrared lasers, pulsed lasers, gas lasers, solid-state lasers, diode lasers, and/or any combinations thereof, e.g., infrared pulsed diode lasers.

In some embodiments, the optical stimulator devices 161, 162 and 163 include optical fiber 32 to provide fiber-optic communication with a laser light source 46 (FIG. 1), e.g., an infrared laser. One or more of the optical stimulator devices 161, 162 and 163 may include a laser emitter (e.g., laser emitter 534 shown in FIG. 5) coupled to the distal end of the optical fiber 32. The laser emitter may have any suitable shape for transmitting and/or focusing light energy including, but not limited to, conical, frustoconical, pyramidal, cylindrical, any other granulated surfaced, combinations thereof, and the like. In some embodiments, the laser light source 46 may include a function generator and optical shutter used to modulate a continuous-wave laser to generate pulsed output.

FIG. 5 shows an end-effector assembly 500 for use with endoscopic surgical procedures. End-effector assembly 500 includes opposing jaw assemblies 510 and 520 which cooperate to effectively grasp tissue therebetween, e.g., for sealing and/or cutting purposes. End-effector assembly 500 includes a nerve stimulator apparatus 560 configured to emit light to stimulate tissue for detection of, or testing and/or monitoring the viability and functionality of nerves. Nerve stimulator apparatus 560 includes a selectively moveable optical device 533, which may be selectively extended and selectively activated.

In some embodiments, as shown in FIG. 5, the end-effector assembly 500 is configured as a unilateral assembly, wherein the jaw member 510 is fixed relative to the shaft 512 and the jaw member 510 pivots about a pivot pin 503 to grasp tissue. Each of the jaw assemblies 510 and 520 includes an outer housing 516 and 526 and an electrically-conductive tissue-engaging surface or sealing plate 512 and 522, respectively. The outer housing 516 and 526 and the sealing plate 512 and 522 shown in FIG. 5 are similar to the outer housing 111 and 121 and the sealing plate 112 and 122, respectively, shown in FIG. 1, and further description of the like elements is omitted in the interests of brevity. One or more of the optical stimulator devices discussed above with respect to FIG. 4 may be associated with the outer housing 516 (and/or outer housing 526).

Optical device 533 includes optical fiber 532, and may include a laser emitter 534 coupled to the distal end of the optical fiber 532. Optical device 533 is communicatively-coupled to a laser light source 546 via the optical fiber 532. The laser emitter 534 may have any suitable shape for transmitting and/or focusing light energy including, but not limited to, conical, frustoconical, pyramidal, cylindrical, any other granulated surfaced, combinations thereof, and the like.

In some embodiments, the optical device 533 is connected to a reciprocatable member 565, which may be operably coupled to a trigger assembly of a surgical instrument (e.g., forceps 10 shown in FIG. 1). In some embodiments, the reciprocatable member 565 may be associated with the outer periphery of the shaft 512. End-effector assembly 500 and the reciprocatable member 565 may be configured such that the optical device 533 may be extended when the jaw assemblies 510 and 520 are in the open or closed position. Alternatively, the optical device 533 may be advanced irrespective of the orientation of the jaw assemblies 510 and 520. End-effector assembly 500 may be configured to allow the optical device 533 to move independently from a knife assembly (not shown) and may be extendable by activation of a trigger assembly (e.g., trigger assembly 70 shown in FIG. 1) or by a separate actuator.

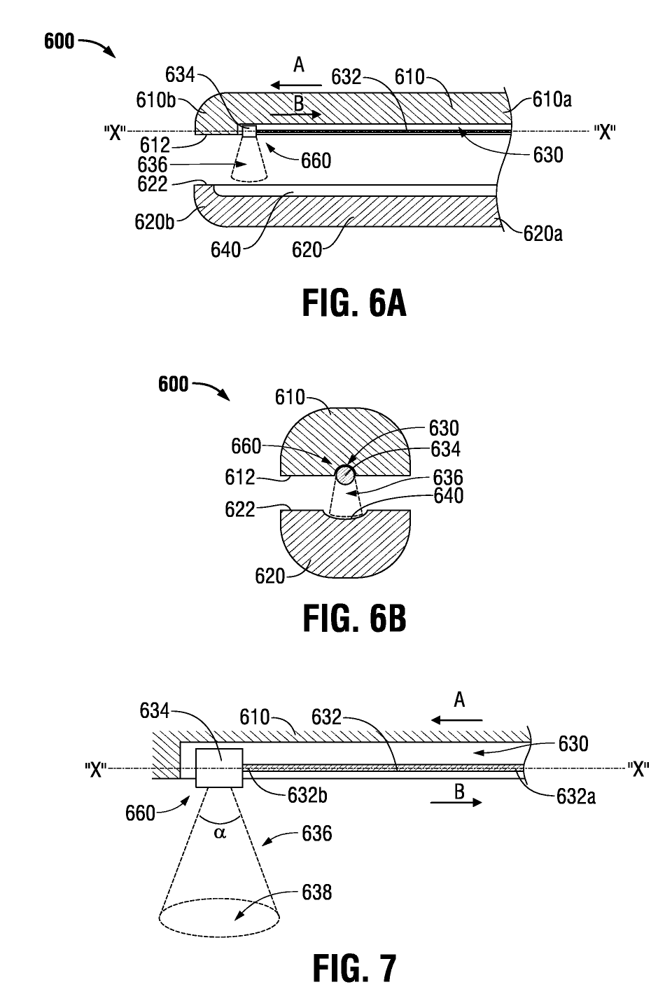

FIGS. 6A, 6B and 7 show an end-effector assembly 600 including a pair of opposing jaw assemblies 610 and 620 and a nerve stimulator apparatus 660 configured to emit light to stimulate tissue for detection of, or testing and/or monitoring the viability and functionality of nerves. Nerve stimulator apparatus 660 includes an optical fiber 632 having proximal and distal ends 632a and 632b, respectively. Jaw assembly 610 includes a channel or groove 630 defined therealong that is configured to receive at least a portion of the optical fiber 632 therein. In some embodiments, as shown in FIGS. 6A, 6B and 7, the nerve stimulator apparatus 660 includes a laser emitter 634 coupled to the distal end 632b of the optical fiber 632. Laser emitter 634 is configured to emit a laser beam into a defined solid angle 636 forming a desired illumination pattern, and may be an “end-firing” laser fiber or a “side-firing” laser fiber. The term “end-firing” as used herein denotes a laser fiber that has the capability to emit a light along a longitudinal axis “X-X” defined by jaw assembly 610. The term “side-firing” as used herein denotes a laser fiber that has the capability to emit light (or any other suitable light energy) in a direction non-parallel to the longitudinal axis “X-X” of jaw assembly 610. Laser emitter 634 may include various components, such as one or more reflective surfaces (e.g., mirrors), one or more optical fibers, one or more lenses, or any other suitable components for emitting and/or dispersing a laser beam.

In some embodiments, laser emitter 634 is configured to emit light into the solid angle 636 that has an outer boundary that may be variable or predetermined. By varying or adjusting the solid angle 636, a laser target area 638 may be adjusted to vary the intensity of the laser light energy illuminating the tissue and the area of the tissue being treated, dissected or cut. Laser target area 638 may define any suitable target shape, for example, but not limited to an ellipse, rectangle, square and triangle. In some embodiments, laser emitter 634 may also be configured to seal and/or cut tissue grasped between the jaw assemblies.

In addition to longitudinal movement of the laser emitter 634 along the longitudinal axis “X-X,” the laser emitter 634 may also be rotated about the axis “X-X” and/or moved laterally (e.g., transverse) with respect thereto. Longitudinal, lateral, and rotational motion of the laser emitter 634 allows for directing light energy in any desired direction to accomplish desired tissue treatment effects.

Reflective groove(s) 640 may be made from a polished metal or a coating may be applied to the jaw member 620 if the jaw member 620 is formed from a non-metal and/or non-reflective material (e.g., plastic). The reflective groove 640 reflects laser light back through the tissue. Laser emitter 634 may receive the reflected laser light and transmit the signal back to the light source for processing. Various types of data may be integrated and calculated to render various outcomes or control tissue treatment based on the transmitted or reflected light.

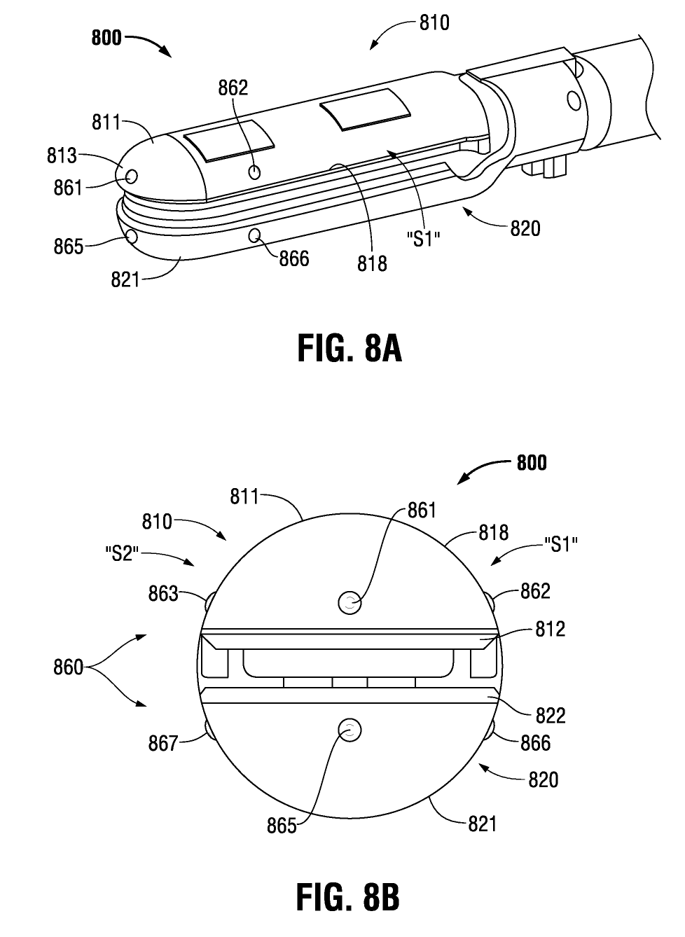

FIGS. 8A and 8B show an end-effector assembly 800 of an endoscopic surgical instrument in accordance with an embodiment of the present disclosure. End-effector assembly 800 generally includes first and second jaw assemblies 810 and 820 disposed in opposing relation relative to one another. End-effector assembly 800 includes a nerve stimulator apparatus 860, including optical stimulator devices associated with both jaw assemblies 810 and 820, configured to emit light to stimulate tissue for detecting, testing and/or monitoring the viability and functionality of nerves.

First and second jaw assemblies 810 and 820 may be either unilateral or bilateral. First and second jaw assemblies 810 and 820 each include an electrically-conductive tissue-engaging surface or sealing plate 812 and 822, respectively, arranged in opposed relation relative to one another and associated with an outer housing 811 and 821, respectively. Each of the outer housings 811 and 821 includes a distal end 813 and 823, respectively, and two lateral side portions (e.g., first lateral side portion “S1” and second lateral side portion “S2” of the housing 811 shown in FIG. 8B). In some embodiments, the outer housings 811 and 821 may be formed, at least in part, of a non-electrically-conductive or substantially non-electrically-conductive material.

In some embodiments, as shown in FIG. 8B, the nerve stimulator apparatus 860 includes a configuration of three optical stimulator devices associated with the first jaw assembly 810 and a configuration of three optical stimulator devices associated with the second jaw assembly 820. First jaw assembly 810 includes a first optical stimulator device 861 disposed at the distal end 813 of the outer housing 811, a second optical stimulator device 862 disposed on the first lateral side portion “S1” of the outer housing 811, a third optical stimulator device 863 disposed on the second lateral side portion “S2” of the outer housing 811. Second jaw assembly 820 includes a fourth optical stimulator device 864 disposed at the distal end 823 of the outer housing 821, a fifth optical stimulator device 865 disposed on the first lateral side portion “S1” of the outer housing 821, a sixth optical stimulator device 866 disposed on the second lateral side portion “S2” of the outer housing 821. End-effector assembly 800 includes optical fiber to provide fiber-optic communication with a laser light source (e.g., light source 546 shown in FIG. 5).

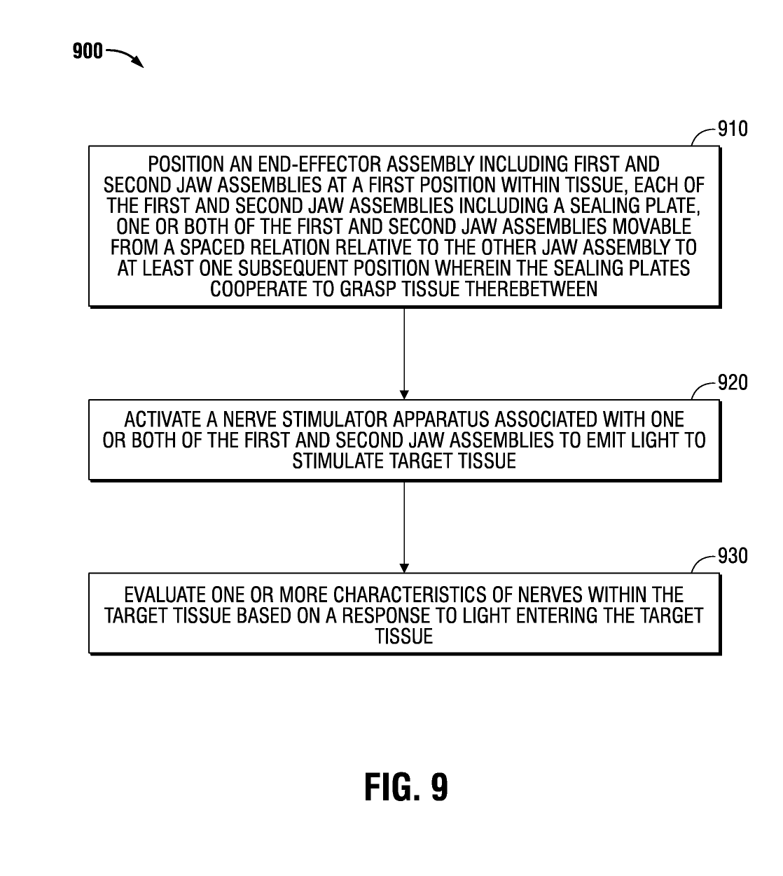

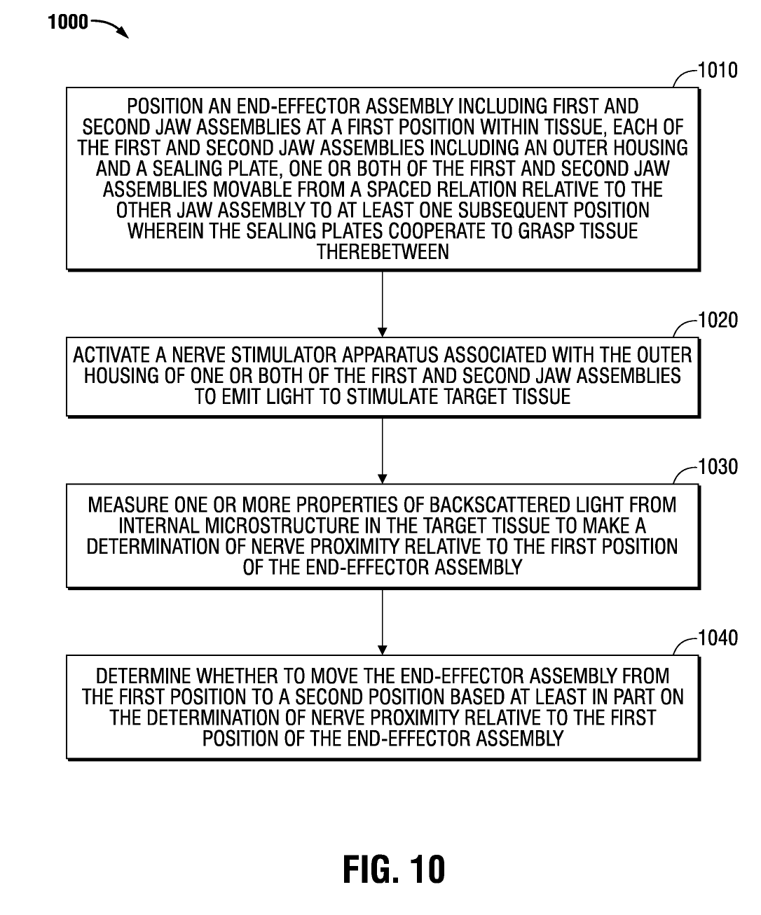

Hereinafter, methods of treating tissue are described with reference to FIGS. 9 and 10. It is to be understood that the steps of the methods provided herein may be performed in combination and in a different order than presented herein without departing from the scope of the disclosure.

FIG. 9 is a flowchart illustrating a method 900 of treating tissue according to an embodiment of the present disclosure. In step 910, an end-effector assembly 100 including first and second jaw assemblies 110 and 120 is positioned at a first position within tissue. Each of the first and second jaw assemblies 110 and 120 includes a sealing plate 112 and 122. One or both of the first and second jaw assemblies 110 and 120 is movable from a spaced relation relative to the other jaw assembly to at least one subsequent position wherein the sealing plates 112 and 122 cooperate to grasp tissue therebetween.

In step 920, a nerve stimulator apparatus 160 associated with the first jaw assembly 110 and/or the second jaw assembly 120 is activated to emit light to stimulate target tissue.

In step 930, one or more characteristics of nerves within the target tissue are evaluated based on a response to light entering the target tissue. In some embodiments, one or more characteristics of nerves within the target tissue include location, viability and functionality of the nerves. In some embodiments, an evaluation of the viability and functionality of nerves may include: detecting and/or monitoring changes in blood pressure, heart rate, and/or breathing rate; detecting muscle contraction and/or twitches; and/or detecting and/or monitoring the release of one or more hormones (and/or other biochemicals). In some embodiments, detecting the location of nerves within the target tissue may include detecting changes in one or more optical properties of the nerves (e.g., fluorescence or absorbance), and may include the use of one or more intraoperative imaging modalities to allow for intraoperative visualization of sensitive structures, e.g., nerves.

FIG. 10 is a flowchart illustrating a method 1000 of treating tissue according to an embodiment of the present disclosure. In step 1010, an end-effector assembly 500 including first and second jaw assemblies 510 and 520 is positioned at a first position within tissue. Each of the first and second jaw assemblies 510 and 520 includes an outer housing 516 and 526 and a sealing plate 512 and 522. One or both of the first and second jaw assemblies 510 and 520 is movable from a spaced relation relative to the other jaw assembly to at least one subsequent position wherein the sealing plates 512 and 522 cooperate to grasp tissue therebetween.

In step 1020, a nerve stimulator apparatus associated with the outer housing of one or both of the first and second jaw assemblies 512 and 522 is activated to emit light to stimulate target tissue.

In step 1030, one or more properties of backscattered light from internal microstructure in the target tissue are measured to make a determination of nerve proximity relative to the first position of the end-effector assembly 500. Examples of properties of backscattered light that may be measured include echo time delay (or reflection) and/or intensity. In some embodiments, the nerve stimulator apparatus may additionally, or alternatively, be activated to emit light to stimulate target tissue for detecting, testing and/or monitoring the viability and functionality of nerves.

In step 1040, a determination is made whether to move the end-effector assembly 500 from the first position to a second position based at least in part on the determination of nerve proximity relative to the first position of the end-effector assembly 500.

The above-described end-effector embodiments including any combination of features of the presently-disclosed nerve stimulator apparatus configured to emit light (e.g., to stimulate tissue for detection of nerves, to stimulate tissue for evaluation of one or more characteristics and/or properties of nerves, and/or to stimulate tissue for testing and/or monitoring of the viability and functionality of nerves) may be used in connection with jaw assemblies of varied geometries, e.g., lengths and curvatures, such that variously-configured jaw assemblies may be fabricated and assembled into various end-effector configurations that include a nerve stimulator apparatus, e.g., depending upon design of specialized surgical instruments.

The above-described bipolar forceps embodiments including a nerve stimulator apparatus configured to emit light to stimulate tissue for determining one or more characteristics and/or properties of nerves may be suitable for use in a variety of procedures and operations. The above-described nerve stimulator apparatus embodiments may be suitable for use for testing and/or monitoring the viability and functionality of nerves, e.g., prior to, during, and/or after the application of energy to tissue during a surgical procedure. The above-described bipolar forceps embodiments including nerve stimulator apparatus may be suitable for utilization with endoscopic surgical procedures and/or hand-assisted, endoscopic and laparoscopic surgical procedures. The above-described bipolar forceps embodiments may be suitable for utilization in open surgical applications.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A forceps, comprising:
a housing;
a shaft including a distal end and a proximal end, the proximal end operatively coupled to the housing;
an end-effector assembly coupled to the distal end of the shaft and including first and second jaw assemblies, each of the first and second jaw assemblies including a sealing plate, at least one of the first and second jaw assemblies movable from a spaced relation relative to the other jaw assembly to at least one subsequent position wherein the sealing plates cooperate to grasp tissue therebetween; and
a nerve stimulator apparatus configured to emit light to stimulate tissue for the evaluation of one or more characteristics of nerves, wherein the nerve stimulator apparatus is disposed in association at least one of the first and second jaw assemblies.

2. The forceps of claim 1, further comprising an optical fiber configured to communicatively couple the nerve stimulator apparatus and a laser light source.

3. The forceps of claim 1, wherein the nerve stimulator apparatus includes an optical fiber having a distal end and a laser emitter coupled to the distal end of the optical fiber.

4. The forceps of claim 1, wherein each of the first and second jaw assemblies further includes an outer housing.

5. The forceps of claim 4, wherein the nerve stimulator apparatus includes at least one optical stimulator device associated with the outer housing of the first jaw assembly.

6. The forceps of claim 5, wherein the at least one optical stimulator device includes at least one infrared pulsed diode laser

7. The forceps of claim 5, wherein the at least one optical stimulator device includes at least one continuous-wave laser.

8. The forceps of claim 5, wherein the at least one optical stimulator device includes at least one ultraviolet laser.

9. The forceps of claim 5, wherein the nerve stimulator apparatus further includes at least one optical stimulator device associated with the outer housing of the second jaw assembly.

10. The forceps of claim 1, wherein the nerve stimulator apparatus includes a selectively moveable optical device.

11. The forceps of claim 10, wherein the optical device is connected to a reciprocatable member, the reciprocatable member operably coupled to a trigger assembly of the forceps.

12. A method of treating tissue, comprising:
positioning an end-effector assembly including first and second jaw assemblies at a first position within tissue, each of the first and second jaw assemblies including a sealing plate, at least one of the first and second jaw assemblies movable from a spaced relation relative to the other jaw assembly to at least one subsequent position wherein the sealing plates cooperate to grasp tissue therebetween;
activating a nerve stimulator apparatus associated with one or both of the first and second jaw assemblies to emit light to stimulate target tissue; and
evaluating at least one characteristic of nerves within the target tissue based on a response to light entering the target tissue.

13. The method of claim 12, wherein the at least one characteristic of nerves within the target tissue includes viability and functionality of nerves.

14. The method of claim 12, wherein the at least one characteristic of nerves within the target tissue includes location of nerves.

15. The method of claim 12, wherein the activating step includes emitting light in the form of optical pulses.

16. The method of claim 12, wherein the activating step includes emitting light in the form of continuous-wave laser irradiation.

17. A method of treating tissue, comprising:
positioning an end-effector assembly including first and second jaw assemblies at a first position within tissue, each of the first and second jaw assemblies including an outer housing and a sealing plate, at least one of the first and second jaw assemblies movable from a spaced relation relative to the other jaw assembly to at least one subsequent position wherein the sealing plates cooperate to grasp tissue therebetween;
activating a nerve stimulator apparatus associated with an outer housing of at least one of the first and second jaw assemblies to emit light to stimulate target tissue;
measuring at least one property of backscattered light from internal microstructure in the target tissue to make a determination of nerve proximity relative to the first position of the end-effector assembly; and
determining whether to move the end-effector assembly from the first position to a second position based at least in part on the determination of nerve proximity relative to the first position of the end-effector assembly.

18. The method of claim 17, wherein the at least one property of backscattered light includes echo time delay and intensity.

19. The method of claim 17, wherein the activating step includes emitting light in the form of optical pulses.

20. The method of claim 17, wherein the activating step includes emitting light in the form of continuous-wave laser irradiation.
